(19) United States
(12) Patent Application Publication
(10) Pub. No.: US 2024/0307627 A1
(43) Pub. Date: Sep. 19, 2024

(54) MEDICAL DEVICE FOR INTRADERMAL INJECTION, METHOD FOR INTRADERMAL INJECTION AND GUIDING DEVICE FOR USE IN SUCH A METHOD

(71) Applicant: KAIRISH INNOTECH Private Ltd., Mumbai (IN)

(72) Inventors: Rishad Kairus Dadachanji, Mumbai (IN); Pratul Prakash Potdar, Nani Daman (IN); Keyurkumarq Arvindbhai Patel, Daman & Diu (IN); Krupal Ashokbhai Chudasma, Ahmedabad (IN)

(73) Assignee: KAIRISH INNOTECH Private Ltd., Mumbai (IN)

(21) Appl. No.: 18/585,789

(22) Filed: Feb. 23, 2024

(30) Foreign Application Priority Data
Mar. 14, 2023 (IN) 202321017135
Apr. 26, 2023 (EP) 23170089.9

(51) Int. Cl. A61M 5/315; A61M 5/32

(52) U.S. Cl. CPC A61M 5/315; A61M 5/3221

(57) ABSTRACT

A medical device for intradermal injection comprises an injection device 6 and a guiding device 1, which is placed on the skin surface 100 of a subject and comprises a tubular guiding device 2 to guide an axial movement of the injection device 6 toward the skin surface 100. A sleeve-shaped guiding portion 32, 33 tightly matches the profile at the distal end of the injection device 6 to prevent a tilting of the injection device 6 at a foremost distal position of the injection device 6 in the tubular guiding device 2. A positive-fit locking member 24 is provided at a side proximal to the sleeve-shaped guiding portion 32, 33, for locking the foremost distal position of the injection device 6 and coupling the injection device 6 to the guiding device 1. Locking of the injection device generates a sound, indicating that the medical device is ready for injection.

### FIELD OF INVENTION

The present invention generally relates to drug delivery devices for injection, and more specifically relates to medical device for intradermal injection of a dosage to a subject, to a method for intradermal injection of a dosage to a subject and to a guiding device (intradermal adapter) for use in such a method.

### BACKGROUND OF INVENTION

Effective drug delivery to the intradermal or shallow subcutaneous (hypodermis) space of a subject requires a precise positioning and guiding of an injection device, such as a syringe, on a skin surface, for precisely defining the penetration depth of an injection needle in the skin surface and the inclination angle of the injection needle during injection. Positioning and guiding an injection device become very challenging particularly if it comes to intradermal injection of a dosage to a subject under relatively small acute angles, such as 15 degrees, and very shallow penetration depths, e.g. of the order of about 2.5 mm, which are to be understood as typical conditions of practice of the device and method of the present application.
U.S. Pat. No. 10,143,810 B2 discloses a needle guide with a base plate and a guiding platform whose angle relative to the base plate can be adjusted. The guiding platform includes a slot that receives a portion of either a needle or a catheter, and guides the needle or catheter for injection.
U.S. Pat. No. 9,655,686 B2 discloses a pistol-shaped guide for guiding the movement of a syringe under a precisely defined angle for injection.
U.S. Pat. No. 8,430,889 B2 discloses a puncture needle holder where the penetration angle of an injection device can be precisely defined.
U.S. Pat. No. 8,083,715 B2 discloses a needle assembly for intradermal injection, comprising an adapter which can adjust the angle of injection, and a tubular guiding cylinder that receives and guides movement of a syringe.
U.S. Pat. No. 10,413,681 B2 discloses an adapter with an injection needle for intradermal injection perpendicular to the skin surface of a subject. The adapter is placed on the skin surface of a subject and coupled with the distal end of an injection syringe.
US 2004/0147901 A1 discloses a device for intradermal injection by means of a syringe, including a tubular housing receiving the whole syringe. The tubular housing has a slanted front end with a base for placement on the skin surface, which defines the angle of penetration of the syringe needle. At the proximal end of the device, a special plunger assembly is provided that is configured to push the plunger rod downward, for injecting a dosage. Slots are provided for defining the penetration depth of the injection needle in the skin.
US 2016/0367766 A1 discloses a device for training staff to properly conduct an injection, which may be an intradermal injection. In certain embodiments, the device includes a tubular housing configured to receive and guide a syringe for injection under a predetermined angle relative to the skin surface of a subject. The front end of the tubular housing is broadened to a planar base surface for placement on the skin surface of a subject. The distal end of the syringe is relatively far away from the skin surface, and measures to control the penetration depth of the needle in the skin surface are not disclosed.

### SUMMARY OF INVENTION

Accordingly, there is a need to provide an enhanced medical device for intradermal injection of a dosage to a subject under repeatable and precisely defined conditions, having a simple and low-cost configuration and enabling a fool-proof handling even by inexperienced or not well-trained staff. Further aspects of the present invention relate to a method for intradermal injection of a dosage to a subject and to a guiding device or intradermal adapter for use in such a method.

According to the present invention there is provided a guiding device for positioning and guiding an injection device for intradermal injection of a dosage to a subject, said injection device comprising a syringe body having a predetermined profile at a distal end thereof and a needle having a distal needle end adapted to penetrate a skin surface of the subject for intradermal injection of the dosage, said guiding device comprising a base having a planar contact surface for alignment with the skin surface of the subject, and a tubular guiding device defining a central axis and configured for guiding an axial movement of the injection device toward the skin surface from a retracted first state where the distal needle end does not penetrate the skin surface and is preferably shielded against the skin surface by a portion of the guiding device to a second state where the distal needle end penetrates the skin surface of the subject. The tubular guiding device defines the central axis and is slanted relative to the contact surface at an acute angle and comprises a distal opening to be passed by the distal needle end for intradermal injection. The tubular guiding device comprises a sleeve-shaped guiding portion that tightly matches the predetermined profile at the distal end of the injection device at least in sections, for preventing a tilting of the injection device about the central axis at or near the foremost distal position of the injection device in the tubular guiding device, and at least one positive-fit locking member, configured for locking the foremost distal position of the injection device in the tubular guiding device in a positive-fit manner and for coupling the injection device to the guiding device in the second state of the injection device. Each of the at least one positive-fit locking member is provided at a side proximal to the sleeve-shaped guiding portion

A further aspect of the present invention relates to a medical device for intradermal injection of a dosage to a subject, comprising an injection device comprising a syringe body having a predetermined profile at a distal end thereof and a needle having a distal needle end adapted to penetrate a skin surface of the subject for intradermal injection of the dosage, and a guiding device defining a central axis and configured for guiding an axial movement of the injection device toward the skin surface from a retracted first state where the distal needle end does not penetrate the skin surface and is preferably shielded against the skin surface by a portion of the guiding device to a second state where the distal needle end penetrates the skin surface of the subject, wherein the guiding device comprises a base having a contact surface, which is planar and configured for alignment with the skin surface of the subject, and a tubular guiding device configured for guiding the axial movement of the injection device toward the skin surface, said tubular guiding device defining the central axis slanted relative to the contact surface at an acute angle and comprising a distal opening to be passed by the distal needle end for intradermal injection. The tubular guiding device is slanted relative to the base at said acute angle and comprises a sleeve-shaped guiding portion that tightly matches the predetermined profile at the distal end of the injection device at least in sections, for preventing a tilting of the injection device about the central axis at or near the foremost distal position of the injection device in the tubular guiding device, and at least one positive-fit locking member, configured for locking the foremost distal position of the injection device in the tubular guiding device in a positive-fit manner and coupling the injection device to the guiding device in the second state of the injection device, wherein the at least one positive-fit locking member is provided at a side proximal to the sleeve-shaped guiding portion.

A further aspect of the present invention relates to a method for intradermal injection of a dosage to a subject by means of an injection device comprising a syringe body having a predetermined profile at a distal end thereof and a needle having a distal needle end, comprising the steps of: providing a guiding device having a guiding device comprising a base having a contact surface, and a tubular guiding device slanted relative to the contact surface at an acute angle and comprising a distal opening, a sleeve-shaped guiding portion that tightly matches the predetermined profile at the distal end of the injection device at least in sections and at least one positive-fit locking member provided at a side proximal to the sleeve-shaped guiding portion; placing the contact surface of the base on the skin surface of the subject, for alignment of the guiding device with the skin surface of the subject;

introducing an injection device into the tubular guiding device; pushing the injection device toward the skin surface along a central axis of the tubular guiding device until the distal needle end extends through the distal opening and projects from the contact surface at said acute angle to penetrate the skin surface of the subject; and injecting the dosage from the syringe body through the needle into the skin surface of the subject. In such a method a penetration depth of the needle in the skin surface of the subject is controlled and a tilting of the injection device about the central axis at or near the foremost distal position of the injection device in the tubular guiding device is prevented by pushing the injection device toward the skin surface along a central axis of the tubular guiding device until the at least one positive-fit locking member locks the foremost distal position of the injection device in the tubular guiding device in a positive-fit manner and the distal end of the injection device is tightly accommodated in a sleeve-shaped guiding portion of the tubular guiding device.

Such a method corresponds to use of a guiding device as disclosed herein under for intradermal injection of a dosage to a subject by means of an injection device comprising a syringe body having a predetermined profile at a distal end thereof and a needle having a distal needle end, in which use the afore-mentioned method steps are carried out.

A further aspect of the present invention relates to a method for controlling intradermal injection of a dosage to a subject by means of an injection device, in particular for controlling the angle and depth of penetration of the needle in the skin surface of a subject for injection.

### Technical Effects

As the sleeve-shaped guiding portion of the tubular guiding device tightly matches the profile at the distal end of the injection device, any tilting of the injection device at or near the foremost distal position in the tubular guiding device can be reliably prevented. Thus, the conditions of the injection device at the stage, when the needle pierces and finally penetrates the skin surface for injection, can be precisely defined. The needle will be guided straight in axial direction and thus pierces and penetrates the skin surface precisely under the acute angle defined by the tubular guiding device without significant deviations, such as those caused by tilting of the injection device.

The sleeve-shaped guiding portion of the tubular guiding device may also serve as a stopping member to delimit the axial movement of the injection device in the tubular guiding device and thus precisely define its foremost distal position and the penetration depth of the needle in the skin surface.

Each of the at least one positive-fit locking member provided at a side proximal to the sleeve-shaped guiding portion reliably locks the foremost distal position of the injection device in the tubular guiding device and couples the injection device to the guiding device. Locking of the injection device by means of the at least one positive-fit locking member may generate a clearly audible noise or sound, e.g. caused by the snapping-back of resilient locking fingers to their relaxed home position, that indicates that the medical device is ready for injection.

In the guiding device according to the present invention, the at least one positive-fit locking member is provided at a relatively large distance away from the sleeve-shaped guiding portion, which further enhances the effect of preventing any tilting of the injection device at or near the foremost distal position in the tubular guiding device.

Moreover, the injection device can only be removed from the skin surface after injection together with the guiding device, as the injection device is coupled to the tubular guiding device by the positive-fit locking member and remains coupled to the tubular guiding device also after the injection is completed. Thus, also during removal of the needle from the skin surface the angle and direction of pulling out the needle from the tissue at the injection site can be precisely defined by the guiding device. Hence, also removal of the needle is very smooth, preventing unnecessary destruction of tissue at the injection site.

By means of a guiding device according to the present invention, medical injection becomes fool-proof and can be executed even by inexperienced and untrained personnel, because both the inclination angle and penetration depth of the needle in the skin surface of a subject is precisely defined by the guiding device at all stages of injection and because the guiding device leaves no possibility of operating the medical device during injection but in a standard and clearly prescribed manner.

### OVERVIEW ON DRAWINGS

The invention will now be described by way of example and with reference to the accompanying drawings, from which further features, advantages and problems to be solved will become apparent. In the drawings:

FIGS. 1a to 1e show a first embodiment of a guiding device for use with an injection device according to the present invention;

FIG. 2a shows an example of an injection device for use with a guiding device according to the present invention, embodied as a syringe;

FIG. 2b shows detail A of FIG. 2a on an enlarged scale;

FIGS. 3a to 3d show the insertion of a syringe into and guiding of a syringe by the guiding device of the first embodiment according to the present invention at four different stages; and

FIG. 3e shows the locking of the syringe in the guiding device of the first embodiment according to the present invention in a foremost distal position of the syringe, for intradermal injection of a dosage to a subject.

In the drawings, the same reference numerals designate identical or substantially equivalent elements or groups of elements.

### DETAILED DESCRIPTION OF PREFERRED EMBODIMENTS

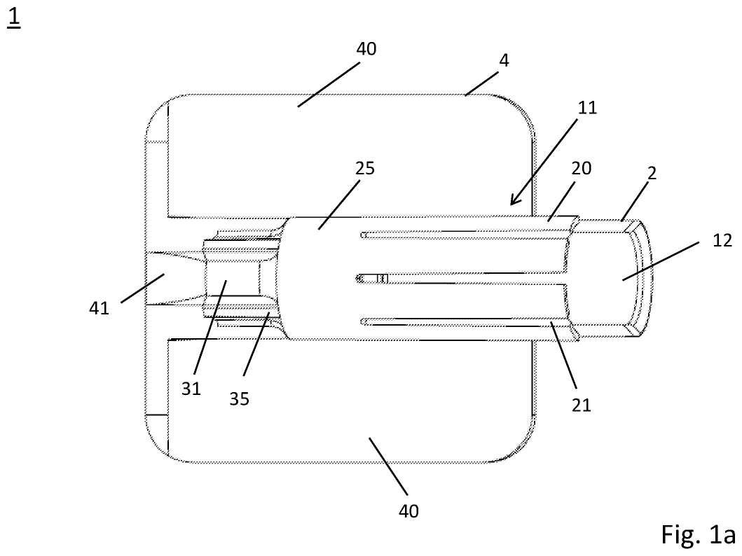

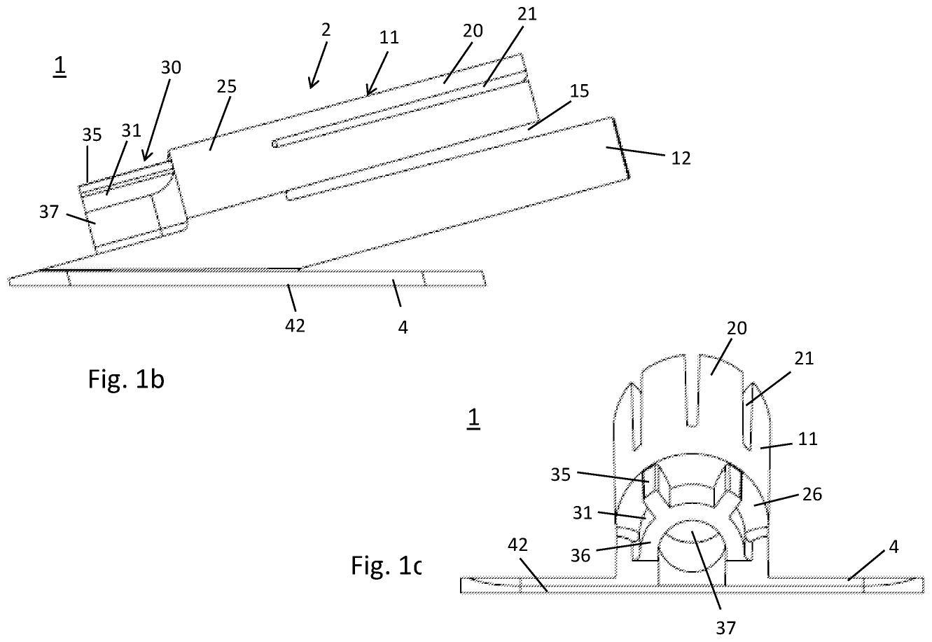

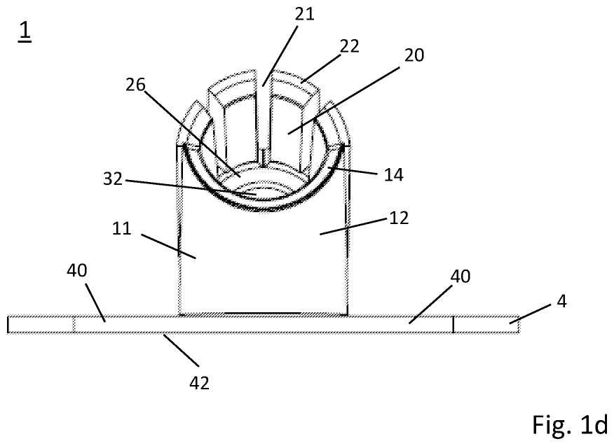

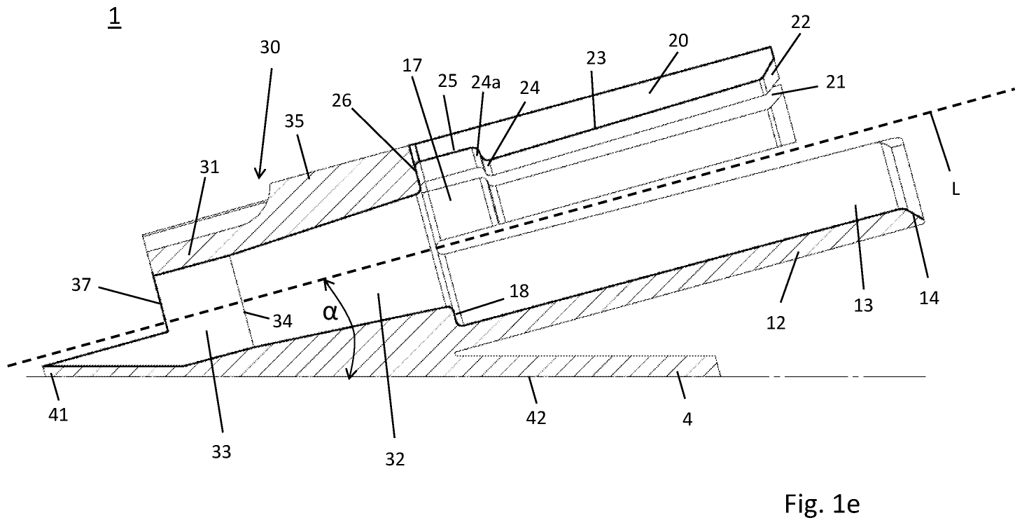

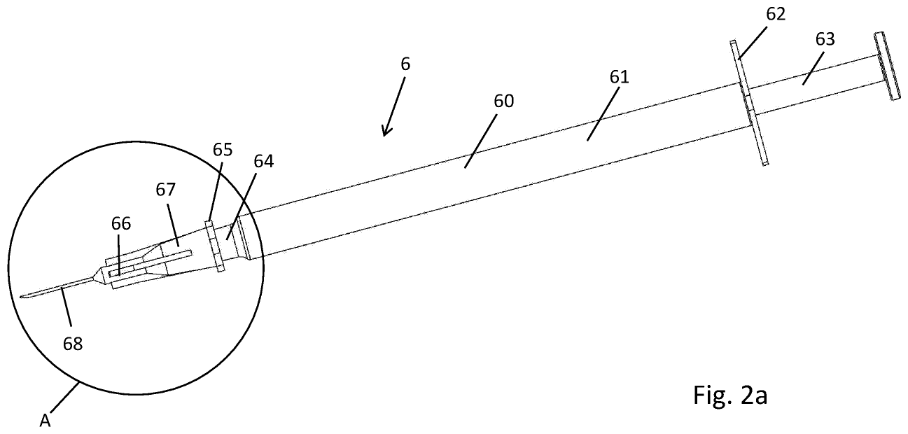

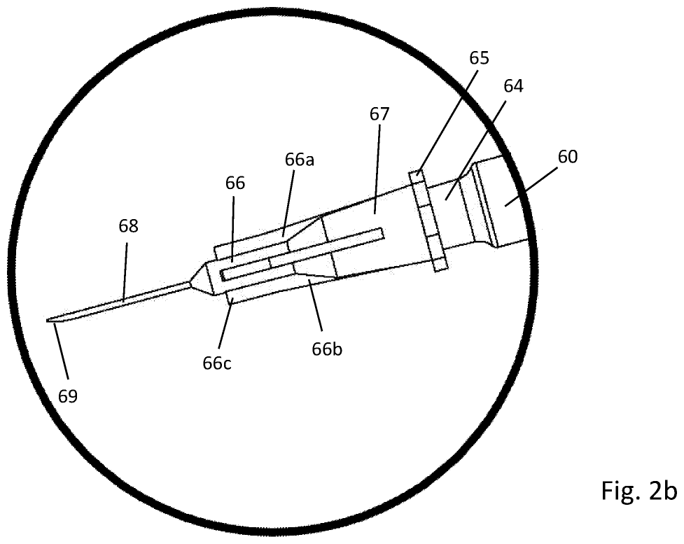

FIGS. 1a to 1e show a guiding device 1 according to the present invention, intended for positioning and guiding an injection device, such as a syringe shown in FIG. 2a, for intradermal injection of a dosage to a subject, and for controlling the angle and depth of penetration of an injection needle in the skin surface of a subject for intradermal injection of a dosage to the subject.

The guiding device is integrally made of a plastic material, e.g. by injection molding, and generally comprises a tubular guiding device 2 and a basis 4, which is preferably integrally formed with the tubular guiding device 2, but may also be connected with the tubular guiding device 2. As shown in FIG. 1e, the tubular guiding device 2 defines a central axis L that is slanted relative to the basis 4 under an acute angle α, which corresponds to the angle under which the needle of an injection device such as a syringe is to penetrate the skin surface of a subject for injection. This angle α may be of the order of 15 degrees, but may be any angle in a wider range e.g. between 5 degrees and 25 degrees, more preferably in a range between 10 degrees and 20 degrees, and even more particularly in a range between 12.5 degrees and 17.5 degrees, depending on the specific application. The base 4 is generally planar and comprises a planar contact surface 42 at a bottom thereof, which is positioned on the skin surface of a subject for injection, e.g. on the inner forearm of a subject, for alignment with the skin surface. For this purpose, the base 4 may comprise two lateral portions 40 each protruding laterally from the tubular guiding device 2 and forming finger support areas configured to supporting the fingers of an operating person when pushing the base 4 with his/her fingers onto the skin surface of the subject for injection. These lateral portions 40 may be slightly bent or bulged, for better conformance e.g. with the inner forearm of a subject.

The inner profile of the tubular guiding device 2 generally corresponds to the outer profile of the injection device to be positioned and guided, and is usually circular, although other profiles, such as square-shaped profiles, may also be envisaged, depending on the outer profile of the injection device. The tubular guiding device 2 includes, at a proximal end thereof, a proximal tubular guiding device 11, which is generally a little wider than the injection device to be guided, to ease insertion of the injection device. For this purpose, the inner profile of the proximal tubular guiding device 11 may be tapered slightly towards the distal end of the guiding device 1. More specifically, the proximal tubular guiding device 11 in the shown embodiment includes a bottom side-wall 12, which is semi-circular in profile and extends in parallel with the central axis L and a plurality of resilient locking fingers 20, which are separated by two longitudinal central slots 15 from the bottom side-wall 12 and the inner surface of which may be tapered slightly towards the distal end of the guiding device 1. The bottom side-wall 12 may be a little longer than the resilient locking fingers 20, to case catching the injection device in the proximal tubular guiding device 2. The plurality of resilient locking fingers 20 are disposed preferably at equiangular intervals around the central axis L and opposite to the bottom side-wall 12 to thereby form an arc-shaped structure for guiding the injection device together with the bottom side-wall 12. The resilient locking fingers 20 are separated by longitudinal slots 21 from each other so that the resilient locking fingers 20 may be spread apart a little, as outlined below in more detail. The stems 25 of the resilient locking fingers 20 are interconnected to form a circular structure together with the distal end of the bottom side-wall 12 at a side distal to the radial steps 18, 26.

Each resilient locking finger 20 preferably comprises a tapered guiding surface 23, which is each tapered toward the distal end of the proximal tubular guiding device 12 and each guiding surface 23 terminates in a locking nose 24 protruding from a respective stem 25 of a resilient locking finger 20. The stem 25 together with a flank of the locking nose 24 and an opposite radial step 26 forms a receptacle 17, which is generally of rectangular shape, if viewed in the cross-sectional view of FIG. 1e, and which is suited to accommodate a protrusion provided at a distal end of the injection device, such as the protrusion 65 at the distal end of the syringe 6 shown in FIGS. 2a and 2b, to thereby lock the injection device to the guiding device 1. The flank 24a of the locking nose 24 may be slanted under a relatively small acute angle relative to a vertical onto the central axis L, whereas the radial step 26 preferably extends perpendicular to the central axis L. The bottom side-wall 12 of the proximal tubular guiding device 11 is relatively stiff and serves as a reference surface for precisely guiding an axial movement of the injection device in the proximal tubular guiding device 11, whereas the resilient locking fingers 20 are less stiff than the bottom side-wall 12 and may be spread apart resiliently during insertion of the injection device into the proximal tubular guiding device 11 to thereby push and resiliently bias the injection device against the bottom side-wall 12, as outlined below in more detail. As shown in FIG. 1e, the receptacle 17 formed by the locking nose 24 and the opposite radial step 26 is preferably formed at the distal end of the proximal tubular guiding device 11 near the stems 25 of the resilient locking fingers 20, but may also be formed at positioned closer to the proximal end of the proximal tubular guiding device 11.

The proximal tubular guiding device 11 is followed by a distal tubular guiding device 30, which generally is less wide and generally tightly matches the predetermined profile at the distal end of the injection device at least in sections, for preventing a tilting of the injection device about the central axis L at or near the foremost distal position of the injection device in the tubular guiding device 30. For this purpose, the tubular guiding device 30 comprises at least one sleeve-shaped guiding portion, of which a first portion directly following the proximal tubular guiding device 11 may be formed as a tapered guiding sleeve 32, as shown in FIG. 1e, which matches a corresponding tapered portion 66b (cf. FIG. 2b) at the distal end of the injection device. Moreover, a cylindrical guiding sleeve 33, which preferably has a constant inner diameter in correspondence to a straight portion 66c (cf. FIG. 2b) at the distal end of the injection device, may also be provided at a side distal to the tapered guiding sleeve 32.

As will become apparent from a comparison of FIG. 1e with the specific profile at the distal end of the injection device 6 shown in FIGS. 2a and 2b, the specific shape of the at least one guiding sleeve 32, 33 at the distal end of the tubular guiding device depends on the specific profile at the distal end of the injection device 6, and the main characteristic of the at least one guiding sleeve 32, 33 is that its profile tightly matches the profile at the distal end of the injection device at least in sections and over a distance in axial direction of the injection that is sufficient to reliably prevent a tilting of the injection device about the central axis L once the distal end of the injection device is accommodated in the at least one guiding sleeve 32, 33 for injection, which corresponds to the position at or near the foremost distal position of the injection device in the tubular guiding device 30. To simplify the present disclosure, in the following it is assumed that the profile at the distal end of the injection device is tapered and includes a straight portion of constant outer diameter as well, as shown in FIGS. 2a and 2b. The present invention shall, however, not be construed to be delimited to such a specific profile at the distal end of the injection device.

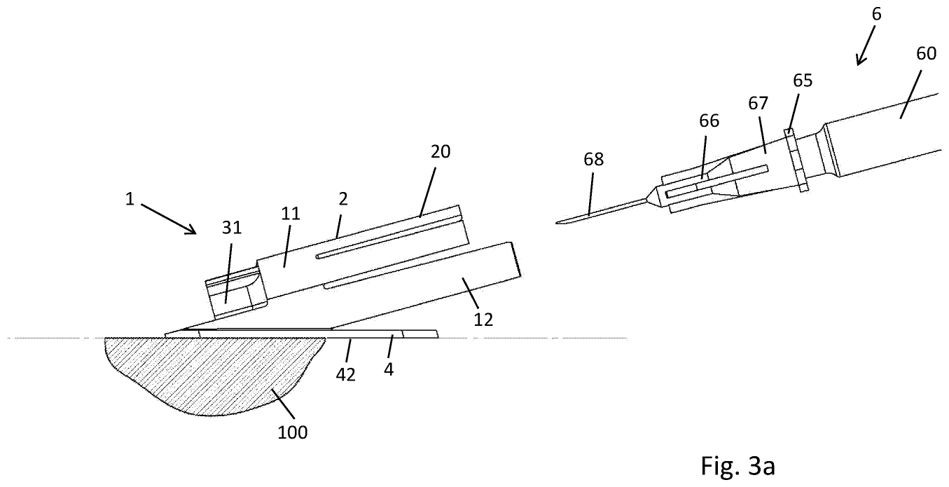

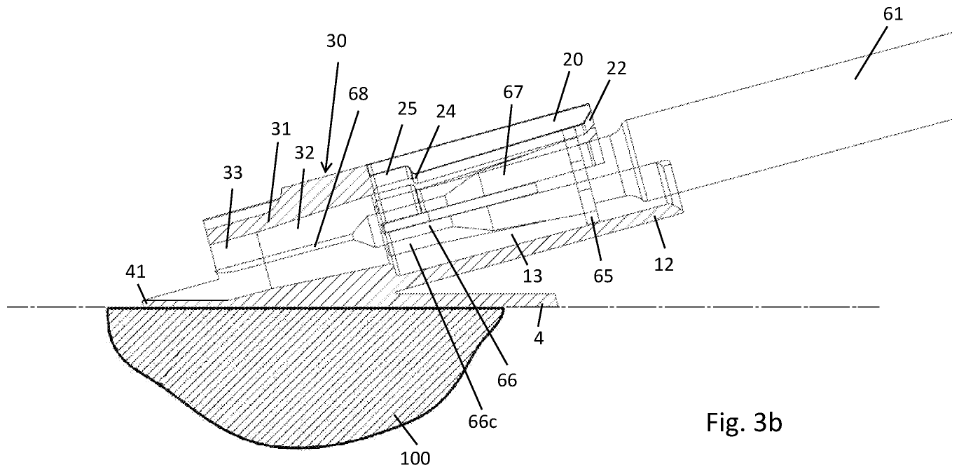

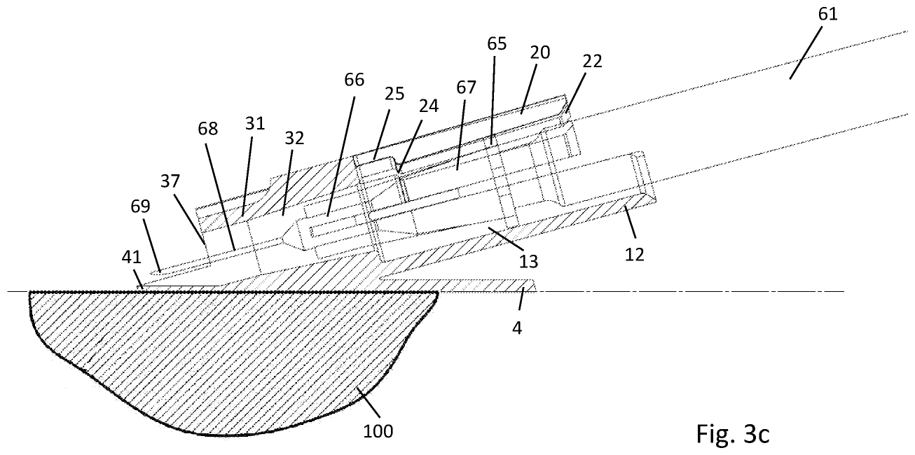

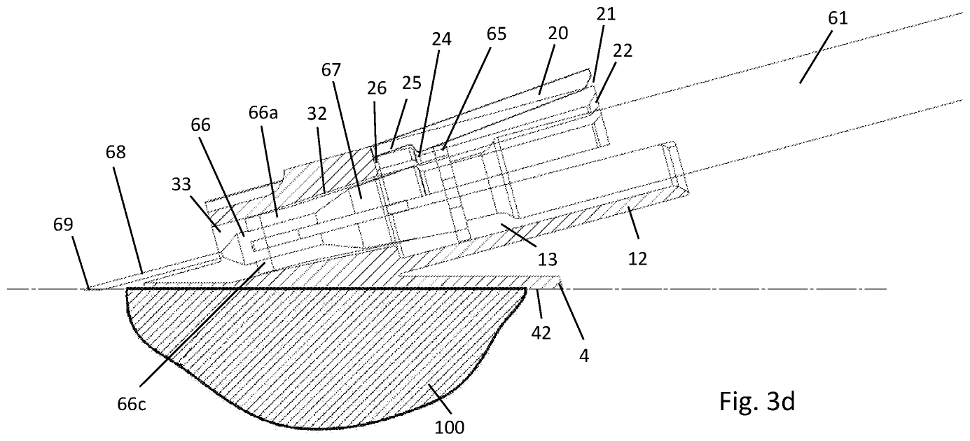

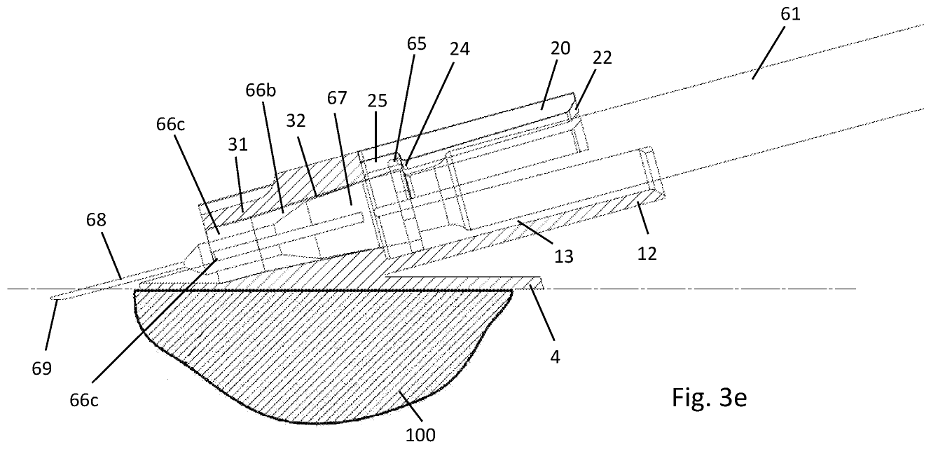

The circular side-wall 31 of the cylindrical guiding sleeve 33 forms a distal opening 37, through which the needle 68 of the injection device passes at or near its foremost axial position in the tubular guiding device 2 for injection, as shown in FIGS. 3d and 3c. However, the front (distal) end of base 4 is long enough to ensure that in a retracted first state where the distal needle end of the injection device does not yet penetrate the skin surface, the distal needle end is shielded against the skin surface by the front (distal) end of base 4, as shown in FIGS. 3a-3c. As shown in FIG. 1c, the circular side-wall 31 of the guiding sleeves 32, 33 is stiffened by axial stiffening ribs 35 and by an arc-shaped structure 36 formed on the outer surface of the side-wall 31 interconnecting the stiffening ribs 35, to substantially prevent any widening of the guiding sleeves 32, 33 upon insertion of the distal end of the injection device, so that the guiding sleeves 32, 33 basically define constant inner profiles during use that tightly match the profile at the distal end of the injection device to be guided and positioned.

In the following, the general configuration of an injection device 6 for use with the above guiding device I will be described with reference to the preferred example of a syringe shown in FIGS. 2a and 2b. It is noted, however, that the present invention shall not be construed to be delimited to such a specific profile at the distal end of the injection device 6 and that the specific configuration of the guiding device 1 can be easily adapted to other profiles at the distal end of the injection device. As shown in FIGS. 2a and 2b, the syringe 6 comprises a cylindrical syringe body 60 of constant outer diameter, which defines a storing chamber 61 for storing a liquid drug or solution to be administered by injection. At the proximal end of the syringe body 60 a finger rest 62 is provided, which enables to control use of the syringe 6 with the forefinger and middle finger and pushing down the plunger rod 63 with the thumb. In this example, a tapered profile is provided at the distal end 64 of syringe body 60. More specifically, a tubular needle mount 66 is mounted to the distal end 64 of syringe body 60, which is formed as a seat fixedly holding the injection needle 68 and establishing a fluid communication between the injection needle 68 and the storage chamber 61. The distal end 69 (needle tip) of injection needle 68 is beveled. The tubular needle mount 66 comprises a conical, tapered portion 67, and at the distal end of this tapered portions 67 a plurality of ribs 66a extend in axial direction, which together define a straight, cylindrical outer profile at the foremost distal end of needle mount 66. At the proximal end of needle mount 66 a circular, radial protrusion 65 is provided, which extends outward and perpendicular to the central axis of syringe 6. The distance between this radial protrusion 65 and the distal needle end 69 is precisely defined and represents an important characteristic for precisely defining the penetration depth of injection needle 68 in the skin surface of a subject during injection. Moreover, the outer profile of the needle mount 66, or more generally of the distal end of the syringe 6 represents an important characteristic for preventing a tilting of the syringe (injection device) about the central axis L in the foremost distal position of the syringe in the tubular guiding device 2.

In the following, the insertion of a syringe 6 into and guiding of a syringe by the guiding device 1 will be described in more detail with reference to FIGS. 3a to 3e. At a first stage, as shown in FIG. 3a, the guiding device 1 is positioned on the skin surface 100 of a subject, by placing the contact surface 42 of base 4 on the skin surface 100 at a desired location, such as the inner forearm of a subject. For a proper alignment of the base 4 with the skin surface 100, the guiding device 1 may be pushed perpendicular to the skin surface 100 during the entire injection process, e.g. by pushing the two lateral portions 40 of base 4 onto the skin surface 100 with two fingers, such as the thumb and fore-finger. Once the guiding device 1 is properly positioned on the skin surface, the syringe 6 is moved towards the guiding device 1, generally along the central axis of the tubular guiding device 2, until the injection needle 68 enters the proximal tubular guiding device 11 and the distal end of syringe 6 is caught by the proximal tubular guiding device 11. In order to case catching the needle mount 66 and radial protrusion 65 of syringe 6, the proximal end surface of the bottom side-wall 12 may be provided with an insertion bevel 14 and the proximal end surfaces of the resilient locking fingers 20 may be provided with insertion bevels 22 as well. Moreover, the bottom side-wall 12 may be a little longer than the locking fingers 20, as shown in FIG. 3a. At this stage, which represents a retracted first stage of syringe 6, the beveled needle end 69 is preferably shielded against the skin surface 100 by a portion of guiding device 1, in particular by the bottom of the cylindrical guiding sleeve 33 and the distal end 41 of base 4.

As shown in FIG. 3b, the radial protrusion 65 of syringe 6 (or more generally the outer surface of a portion of syringe 6) continues sliding along the tapered guiding surfaces 23 of the resilient locking fingers 20 and the bottom side-wall 12 towards the distal end of the guiding device, until the cylindrical portion 66c of needle mount 66 finally enters the cylindrical guiding sleeve 33 (cf. FIG. 3d), so that the syringe 6 starts to be aligned more or less perfectly with the central axis L of tubular guiding device 2. At the stage shown in FIG. 3b, which still represents a retracted first stage of syringe 6, the beveled needle end 69 preferably remains shielded against the skin surface 100 by a portion of guiding device 1, in particular by the bottom of the cylindrical guiding sleeve 33 and the distal end 41 of base 4. At this stage, the resilient locking fingers 20 start to be spread apart due to cooperation with the radial protrusion 65 of syringe 6, and the syringe 6 is pushed perpendicular to central axis L towards the bottom side-wall 12 by the resilient locking fingers 20. The bottom side-wall 12 thus serves as a reference surface at all stages of injection, and the resilient locking fingers 20 serve for centering the syringe 6. Moreover, the syringe 6 may still tilted by a relatively small angle only about the central axis of tubular guiding device 2, because the gap between the side-wall 31 of tapered guiding sleeve 32 and the cylindrical portion 66c of needle mount 66 is relatively narrow already.

As shown in FIG. 3c, finally the beveled needle end 69 leaves the cylindrical guiding sleeve 33 and passes the distal opening 37. The radial protrusion 65 of syringe 6 (or more generally the outer surface of a portion of syringe 6) continues sliding along the tapered guiding surfaces 23 of the resilient locking fingers 20 and the bottom side-wall 12, and the gap between the side-wall 31 of tapered guiding sleeve 32 and the cylindrical portion 66c of needle mount 66 gradually becomes narrower. As the syringe 6 is further moved in axial direction, the resilient locking fingers 20 are increasingly spread apart, until the stage shown in FIG. 3d is finally reached.

At this stage, the resilient locking fingers 20 are spread apart significantly by the radial protrusion 65, to thereby push the radial protrusion 65 and thus the distal end of syringe 6 towards the bottom side-wall 12 of the proximal tubular guiding device 11. At this stage, the cylindrical portion 66c of needle mount 66 is already accommodated in the cylindrical guiding sleeve 33, the profile of which tightly matches the outer profile of cylindrical portion 66c of needle mount 66 is. Hence, at this stage, where the beveled needle end 69 starts piercing the skin surface, the syringe is already perfectly centered in tubular guiding device 2 and aligned with central axis L, and there exists no play anymore (or only a very little play) for tilting the syringe 6 about the central axis of the guiding device. At this stage, the radial protrusion 65 has nearly reached the locking noses 24.

When the syringe 6 is moved further in axial direction, the beveled needle end 69 finally penetrates the skin surface under the acute angle α defined by the tubular guiding device 2 and the radial protrusion 65 continues spreading the resilient locking fingers 20 further apart. The further axial movement of syringe 6 is precisely guided along central axis L, as the cylindrical portion 66c of needle mount 66 is tightly accommodated in cylindrical guiding sleeve 33, without radial play. Moreover, the radial protrusion 65 and thus the distal portion of syringe 6 is pushed towards the bottom side-wall 12 of the proximal tubular guiding device 11 by the resilient locking fingers 20. Hence, any tilting of syringe 6 about central axis L is reliably prevented.

Finally, the radial protrusion 65 starts sliding over the locking noses 24 and along the flanks 24a of locking noses 24. At this stage, the resilient locking fingers 20 snap back to their relaxed home position shown in FIG. 3e. This snapping back of the resilient locking fingers 20 results in a characteristic audible clicking-noise or sound that will be clearly perceived by the person operating syringe 6 and guiding device 1, and represents a clear and unambiguous signal to that person indicating that the syringe 6 has reached its foremost distal position and is ready for injection of the dosage to the subject by pushing the plunger rod 63 into syringe body 60.

At this stage, the tapered portion 67 of needle mount 66 completely abuts against the tapered inner surface of tapered guiding sleeve 32. As the flanks 24a of locking noses 24 may be slightly tilted relative to a vertical onto central axis L, the radial protrusion 65 and hence the distal end of syringe 6 may be resiliently biased towards the tapered inner surface of tapered guiding sleeve 32. This foremost distal position of syringe 6 in tubular guiding device precisely defines the penetration depth of needle 68 in the skin surface 100. More specifically, this foremost distal position is defined by the cooperation with the predetermined outer profile at the distal end of the syringe 6 with the tapered and/or cylindrical guiding sleeve 32, 33. At this stage, the beveled needle end 69 projects beyond the bottom contact surface 42 of base 4. As an alternative, a semi-circular aperture may be formed at the front portion of base 4, in the area designated by reference numeral 41 in FIG. 1a, and the needle end 69 may pass this aperture for piercing and finally penetrating the skin of the subject.

In order to control the angular orientation of the beveled needle end 69, an indexing feature may be provided at the distal end of the tubular guiding device 2, in particular at the distal end of the tapered and/or cylindrical guiding sleeve 32, 33, such as a protrusion or rib, which cooperates with a corresponding feature on the outer surface of the syringe 6, in particular at the outer surface of the tubular needle mount 66. Such an indexing feature may be formed in particular as a guiding rib formed on the outer surface of the tubular needle mount 66, for determining the orientation of the beveled needle end 69 by cooperation with the corresponding indexing feature of the tubular guiding device 2.

The medical device consisting of guiding device 1 and syringe 6 can be operated easily by a person, preferably by placing and positioning the guiding device 1 on a skin surface 100 with one hand and then inserting syringe 6 into the tubular guiding device 2 and operating syringe 6 with the other hand.

By means of the guiding device 1 medical injection becomes fool-proof and can be executed even by inexperienced and untrained personnel, because both the inclination angle and penetration depth of injection needle 68 in the skin surface 100 is precisely defined by the guiding device 1 and because the guiding device 1 leaves no possibility of operating the medical device during injection in a different manner as outlined above.

As will become apparent to the skilled person when studying the above description, in the embodiment described above the tapered guiding sleeve 32 serves as a stopping member defining the foremost distal position of the syringe 6 in the tubular guiding device 2, namely by abutment of tapered portion 67 of needle mount 66 against the inner surface of tapered guiding sleeve 32. Once the distal end of the injection device 6 is accommodated in the tapered guiding sleeve 32, a further axial movement of the injection device is prevented. Thus, the penetration depth of injection needle 68 in the skin surface 100 is precisely defined.

As the injection device 6 is locked to the guiding device 1 in this foremost axial position by the at least one positive-fit locking member, namely in the afore-mentioned example by means of the locking noses 24, the injection device 6 can be removed from the skin surface 100 (injection site) after injection of the dosage to the subject only together with the guiding device 1, because the injection device 6 is firmly coupled to the guiding device 1, being locked by the locking noses 24. For this purpose, the guiding device 1 together with the injection device 6 may be pulled a little backward from the injection site and then pulled away from the skin surface 100 along the axial direction defined by the central axis L, to ensure a smooth removal of the needle 68 without destroying tissue at the injection site.

After complete removal of the guiding device 1 from the skin surface 100, the injection device 6 can then be separated from the guiding device 1 by pulling the injection device 6 out of the tubular guiding device 2, thereby either overcoming the holding force exerted by the at last one positive-fit locking member or separating the distal end of the injection device 6, such as the tubular needle mount 66 disclosed above, from the injection device 6 and leaving the distal end together with the needle 68 locked to the tubular guiding device 2, for disposal.

According to the present invention, tolerances in the outer dimensions at the distal end of syringe 6 can be compensated, because the locking noses 24 of the resilient locking fingers 20 resiliently bias radial protrusion 65 and hence the distal end of syringe 6 against the tapered guiding sleeve 32, wherein the tapered guiding sleeve 32 serves as a stopping member for defining the foremost axial position of syringe 6. The, the penetration depth of the needle 68 in the skin surface 100 may be defined even more precisely. At the same time, the tapered guiding sleeve 32 tightly matches the predetermined profile at the distal end of the injection device 6 at least in sections, so that a tilting of the injection device 6 about the central axis L at or near the foremost distal position of the injection device 6 in the tubular guiding device 2 is reliably prevented. For this purpose, the tapered guiding sleeve 32 extends in axial direction over a certain distance allowing to precisely define and maintain the position of the distal end of the injection device 6 in radial direction. Preferably, the tapered guiding sleeve 32 and the cylindrical guiding sleeve 33 completely surround the distal end of syringe 6 at the foremost, locked position. It may be sufficient, however, if the tapered guiding sleeve 32 and the cylindrical guiding sleeve 33 about against the distal end of syringe 6 in sections, e.g. by means of axial ribs defining a contour tightly matching the predetermined profile at the distal end of syringe 6, e.g. of the tubular needle mount 66, as long as the surfaces of such axial ribs together span a circular surface or contour tightly matching the predetermined profile at the distal end of syringe 6.

Of course, there exist many other options for implementing a stopping member preventing a further axial movement of the injection device (syringe) in the tubular guiding device beyond the stopping member. As an example, a stopping member may be formed as a protrusion provided in the sleeve-shaped guiding portion 32, 33 or elsewhere inside the tubular guiding device to cooperate with a corresponding contour, such as a radial protrusion or depression formed at a corresponding position on the outer surface of the injection device (syringe) 6, in a positive-fit manner. Generally, a stopping member may be implemented by means of frictional engagement and/or positive-fit. Frictional engagement may be between an outer contour at the distal end of the injection device (syringe) 6 and a corresponding profile formed inside the tubular guiding device 2, preferably at a foremost distal end thereof, such as the tapered guiding sleeve 32 and/or the cylindrical guiding sleeve 33. A proper frictional pairing between the material at the distal end of injection device (syringe) 6 and the material of the corresponding section of the tubular guiding device 2 may assist in establishing such a frictional engagement to define the foremost distal position of injection device (syringe) 6 inside the tubular guiding device 2.

While in the preferred embodiment outlined above, the radial protrusion 65 is part of a tubular needle mount 66 that is a member separate to the injection device (syringe) 6, it will become apparent to the skilled person when studying the above description, that such a radial protrusion, or also a depression, may be also be formed as an integral part of the injection device (syringe) 6. Such a radial protrusion or depression may also be part of a separate member, such as a sleeve, to be mounted near the distal end of the injection device (syringe) 6 at a predetermined location by imposing such a separate member on the distal end of the injection device (syringe) 6 or by clamping it at the injection device (syringe) 6 at a proper position.

As will become apparent to the skilled person when studying the above description, a locking feature for locking the injection device to the guiding device may also be implemented in a different manner as disclosed above by cooperation of the radial protrusion 65 with the locking noses 24 of the resilient locking fingers 20 in a positive-fit manner. In particular, the locking noses may be implemented as protrusions protruding radially from the inner surfaces of the resilient locking fingers 20 and cooperating with a semi-circular or circular groove formed on the outer surface of the injection device, or as indentations formed on the inner surfaces of the resilient locking fingers 20 and cooperating with a semi-circular or circular protrusion formed on the outer surface of the injection device.

As will become apparent to the skilled person when studying the above description, a medical device for intradermal injection of a dosage to a subject consists of an injection device and a guiding device as outlined above, which may be delivered to customers as a set consisting of two separate members or as individual members. Preferably, the injection device is a syringe. However, the present invention shall not be construed to be delimited to use of syringes only.

As will become apparent to the skilled person when studying the above description, a further aspect of the present invention relates to a guiding device (intradermal adapter) as outlined above, for use together with an injection device having a corresponding profile at least at a distal end thereof.

As will become apparent to the skilled person when studying the above description, a further aspect of the present invention relates to the use of a guiding device (intradermal adapter) as outlined above, together with an injection device having a corresponding profile at least at a distal end thereof, for intradermal injection of a dosage to a subject.

As will become apparent to the skilled person when studying the above description, a further aspect of the present invention relates to a method for intradermal injection of a dosage to a subject by means of an injection device, such as a syringe, and a corresponding guiding device (intradermal adapter), as outlined above.

While the preferred embodiments of the present invention have been described so as to enable one skilled in the art to practice the medical device, intradermal adapter and method for intradermal injection of a dosage to a subject of the present invention, it is to be understood that variations and modifications may be employed without departing from the concept and intent of the present invention as defined in the appended claims. Accordingly, the preceding description is intended to be exemplary and should not be used to limit the scope of the invention. The scope of the invention should be determined only by reference to the appended claims.

### LIST OF REFERENCE NUMERALS

1 guiding device
2 tubular guiding device
4 base
6 injection device/syringe
10 medical device for intradermal injection
11 proximal tubular guiding device
12 bottom side wall
13 proximal guiding sleeve
14 insertion bevel
15 central slot
17 receptacle
18 radial step
20 locking finger
21 longitudinal slot
22 insertion bevel
23 tapered guiding surface
24 locking nose
24a flank of locking nose 24
25 stem
26 radial step
30 distal tubular guiding device
31 side wall
32 tapered guiding sleeve
33 cylindrical guiding sleeve
34 distal end of tapered guiding sleeve 32
35 stiffening rib
36 arc
37 distal opening
40 lateral portion of base 4
41 distal end of base 4
42 contact surface of base 4
60 syringe body
61 storing chamber
62 finger rest
63 plunger rod
64 distal end of syringe body
65 protrusion
66 needle mount
66a rib
66b tapered portion of rib 66a
66c straight portion of rib 66a
67 tapered portion of needle mount
68 injection needle
69 beveled needle end
100 skin surface
A detail in FIG. 2a
L central axis
α angle of inclination

What is claimed is:

1. A guiding device for positioning and guiding an injection device for intradermal injection of a dosage to a subject, said injection device comprising a syringe body having a predetermined profile at a distal end thereof and a needle having a distal needle end adapted to penetrate a skin surface of the subject for intradermal injection of the dosage, said guiding device comprising:
a base having a contact surface for alignment with the skin surface of the subject, and
a tubular guiding device configured for guiding an axial movement of the injection device toward the skin surface from a retracted first state where the distal needle end does not penetrate the skin surface and is shielded against the skin surface by a portion of the guiding device to a second state where the distal needle end penetrates the skin surface of the subject, said tubular guiding device defining the central axis slanted relative to the contact surface at an acute angle and comprising a distal opening to be passed by the distal needle end for intradermal injection; wherein
the contact surface of the base is planar, and
the tubular guiding device is slanted relative to the base at said acute angle; and wherein
the tubular guiding device comprises:
a sleeve-shaped guiding portion that tightly matches the predetermined profile at the distal end of the injection device at least in sections, for preventing a tilting of the injection device about the central axis at or near the foremost distal position of the injection device in the tubular guiding device, and
at least one positive-fit locking member configured for locking the foremost distal position of the injection device in the tubular guiding device in a positive-fit manner and for coupling the injection device to the guiding device in the second state of the injection device, wherein
the at least one positive-fit locking member is provided at a side proximal to the sleeve-shaped guiding portion.

2. The guiding device as claimed in claim 1, wherein the at least one positive-fit locking member is configured to resiliently bias the injection device toward the foremost distal position of the injection device in the tubular guiding device.

3. The guiding device as claimed in claim 1, wherein
the sleeve-shaped guiding portion comprises a tapered guiding sleeve near a distal end of the tubular guiding device to define the foremost distal position of the injection device in the tubular guiding device by at least one of frictional engagement and positive-fit.

4. The guiding device as claimed in claim 1, wherein the tubular guiding device further comprises a proximal tubular guiding device disposed at a side proximal to the sleeve-shaped guiding portion and comprising at least one resilient locking finger, wherein each positive-fit locking member is provided at a respective resilient locking finger.

5. The guiding device as claimed in claim 4, wherein each positive-fit locking member is formed as a locking nose or locking depression integrally formed with the respective resilient locking finger and configured to cooperate with a corresponding protrusion or depression provided on an outer surface of the injection device.

6. The guiding device as claimed in claim 4, wherein the proximal tubular guiding device is formed by a bottom side-wall extending in parallel with the central axis and a plurality of resilient locking fingers disposed at equiangular intervals around the central axis and opposite to the bottom side-wall, each resilient locking finger comprising a tapered guiding surface, which is tapered toward the respective positive-fit locking member.

7. The guiding device as claimed in claim 5, wherein each resilient locking finger comprises a tapered guiding surface tapered toward the distal end of the proximal tubular guiding device and terminating in the locking nose protruding from a respective stem of the respective resilient locking finger.

8. The guiding device as claimed in claim 7, wherein the stem of the respective resilient locking finger together with a flank of the respective locking nose and a radial step opposite along a direction of the central axis forms a receptacle of a rectangular shape, if viewed in a cross-sectional view.

9. The guiding device as claimed in claim 3, wherein the tubular guiding device further comprises a cylindrical guiding sleeve provided at a distal end of the tapered guiding sleeve, configured to cooperate with a distal end of the injection device at or near the foremost distal position of the injection device in the tubular guiding device, for centering the injection device in the tubular guiding device when the distal needle end penetrates the skin surface of the subject for intradermal injection.

10. The guiding device as claimed in claim 9, wherein the tubular guiding device further comprises a proximal tubular guiding device disposed at a side proximal to the sleeve-shaped guiding portion and comprising at least one resilient locking finger, wherein each positive-fit locking member is provided at a respective resilient locking finger and formed as a locking nose or locking depression integrally formed with the respective resilient locking finger and configured to cooperate with a corresponding protrusion or depression provided on an outer surface of the injection device.

11. A medical device for intradermal injection of a dosage to a subject, comprising
an injection device comprising a syringe body having a predetermined profile at a distal end thereof and a needle having a distal needle end adapted to penetrate a skin surface of the subject for intradermal injection of the dosage; and
a guiding device defining a central axis and configured for guiding an axial movement of the injection device toward the skin surface from a retracted first state where the distal needle end does not penetrate the skin surface and is shielded against the skin surface by a portion of the guiding device to a second state where the distal needle end penetrates the skin surface of the subject; wherein
the guiding device comprises
a base having a contact surface, which is planar, for alignment with the skin surface of the subject, and
a tubular guiding device configured for guiding the axial movement of the injection device toward the skin surface, said tubular guiding device defining the central axis slanted relative to the contact surface at an acute angle and comprising a distal opening to be passed by the distal needle end for intradermal injection, wherein
the tubular guiding device is slanted relative to the base at said acute angle and comprises:
a sleeve-shaped guiding portion that tightly matches the predetermined profile at the distal end of the injection device at least in sections, for preventing a tilting of the injection device about the central axis at or near the foremost distal position of the injection device in the tubular guiding device, and
at least one positive-fit locking member configured for locking the foremost distal position of the injection device in the tubular guiding device in a positive-fit manner and for coupling the injection device to the guiding device in the second state of the injection device, wherein
the at least one positive-fit locking member is provided at a side proximal to the sleeve-shaped guiding portion.

12. The medical device as claimed in claim 11, wherein the at least one positive-fit locking member is configured to resiliently bias the injection device toward the foremost distal position of the injection device in the tubular guiding device.

13. The medical device as claimed in claim 11, wherein
the sleeve-shaped guiding portion comprises a tapered guiding sleeve near a distal end of the tubular guiding device to define the foremost distal position of the injection device in the tubular guiding device by at least one of frictional engagement and positive-fit.

14. The medical device as claimed in claim 11, wherein the tubular guiding device further comprises a proximal tubular guiding device disposed at a side proximal to the sleeve-shaped guiding portion and comprising at least one resilient locking finger, wherein
each positive-fit locking member is provided at a respective resilient locking finger and
each positive-fit locking member is formed as a locking nose or locking depression integrally formed with the respective resilient locking finger and configured to cooperate with a corresponding protrusion or depression provided on an outer surface of the injection device.

15. The medical device as claimed in claim 14, wherein the proximal tubular guiding device is formed by a bottom side-wall extending in parallel with the central axis and a plurality of resilient locking fingers disposed at equiangular intervals around the central axis and opposite to the bottom side-wall, each resilient locking finger comprising a tapered guiding surface, which is tapered toward the respective positive-fit locking member.

16. The medical device as claimed in claim 15, wherein each resilient locking finger comprises a tapered guiding surface tapered toward the distal end of the proximal tubular guiding device and terminating in the locking nose protruding from a respective stem of the respective resilient locking finger, wherein the stem of the respective resilient locking finger together with a flank of the respective locking nose and a radial step opposite along a direction of the central axis forms a receptacle of a rectangular shape, if viewed in a cross-sectional view.

17. The medical device as claimed in claim 15, wherein the tubular guiding device further comprises a cylindrical guiding sleeve provided at a distal end of the tapered guiding sleeve, configured to cooperate with a distal end of the injection device at or near the foremost distal position of the injection device in the tubular guiding device, for centering the injection device in the tubular guiding device when the distal needle end penetrates the skin surface of the subject for intradermal injection.

18. The medical device as claimed in claim 11, wherein the injection device further comprises a tubular needle mount mounted to the distal end of the syringe body and the tubular needle mount is formed as a seat fixedly holding the needle and establishing a fluid communication between the needle and a storing chamber of the syringe body, wherein the tubular needle mount comprises a tapered portion and a portion having a cylindrical outer profile disposed at a side distal to the tapered portion.

19. In a method for intradermal injection of a dosage to a subject by means of an injection device comprising a syringe body having a predetermined profile at a distal end thereof and a needle having a distal needle end, comprising the steps of:
providing a guiding device having a guiding device comprising a base having a contact surface, and a tubular guiding device slanted relative to the contact surface at an acute angle and comprising a distal opening, a sleeve-shaped guiding portion that tightly matches the predetermined profile at the distal end of the injection device at least in sections and at least one positive-fit locking member provided at a side proximal to the sleeve-shaped guiding portion;
placing the contact surface of the base on the skin surface of the subject, for alignment of the guiding device with the skin surface of the subject;
introducing an injection device into the tubular guiding device;
pushing the injection device toward the skin surface along a central axis of the tubular guiding device until the distal needle end extends through the distal opening and projects from the contact surface at said acute angle to penetrate the skin surface of the subject; and
injecting the dosage from the syringe body through the needle into the skin surface of the subject;
controlling a penetration depth of the needle in the skin surface of the subject and preventing a tilting of the injection device about the central axis at or near the foremost distal position of the injection device in the tubular guiding device by
pushing the injection device toward the skin surface along a central axis of the tubular guiding device until the at least one positive-fit locking member locks the foremost distal position of the injection device in the tubular guiding device in a positive-fit manner and the distal end of the injection device is tightly accommodated in a sleeve-shaped guiding portion of the tubular guiding device.

20. The method for intradermal injection as claimed in claim 19, further comprising:
resiliently biasing the at least one positive-fit locking member toward the foremost distal position of the injection device in the tubular guiding device.
